(19) United States
(12) Patent Application Publication
(10) Pub. No.: US 2016/0174821 A1
(43) Pub. Date: Jun. 23, 2016

(54) Disposable Protective Sleeve for Equine Endoscope

(71) Applicants: Heidi Homze, Elizabeth, CO (US); Glenn Russell Thompson, Colts Neck, NJ (US)

(72) Inventors: Heidi Homze, Elizabeth, CO (US); Glenn Russell Thompson, Colts Neck, NJ (US)

(21) Appl. No.: 14/578,486

(22) Filed: Dec. 22, 2014

(51) Int. Cl. A61B 1/00; A61B 10/00

(52) U.S. Cl. CPC A61B 1/00142; A61B 1/00128; A61B 10/0045; A61B 2503/40

(57) ABSTRACT

A disposable protective sleeve for an equine endoscope made from thin, flexible plastic used to cover the endoscope during examination of an equine patient in order to prevent cross contamination between equine patients. The distal end may optionally have a small pocket for collection of samples, such as mucous, for submission to a laboratory for testing.

### BACKGROUND OF THE INVENTION

The present invention pertains to the field of prevention of spread of disease between equine patients. In the field of human medicine, endoscopes are routinely sterilized after use with each patient in order to prevent the spread of disease from one patient to the next. Endoscopes are sterilized both inside and out, and this involves cleaning of any debris, disinfection by immersion of the scope in a high-level disinfectant, rinsing with water, and drying. (Disinfection of Healthcare Equipment, Guideline for Disinfection and Sterilization in Healthcare Facilities, 2008, Centers for Disease Control and Prevention).

Time and cost constraints on the equine practitioner usually prevent conducting such intricate sterilization procedures between patients. The current method of sterilizing equine endoscopes between patients often consists of wiping the outer surface of the endoscope with a disinfectant such as ethyl alcohol or isopropyl alcohol. This is insufficient for several reasons. Alcohol must come into contact with all surfaces and requires wet contact time to be effective in killing bacteria, which is difficult because it rapidly evaporates. It also does not kill bacterial spores, some protozoa, certain non-enveloped viruses nor prions, and cannot penetrate protein-rich materials. Alcohol also can damage instruments when used over time. The CDC does not recommend the use of alcohols for sterilizing medical materials used on human patients (Chemical Disinfectants, Guideline for Disinfection and Sterilization in Healthcare Facilities, 2008, Centers for Disease Control and Prevention). Additionally, the use of alcohol or other surface disinfectants on endoscopes can be very inconvenient for equine veterinarians who often work in adverse conditions, such as cold weather. For these reasons, sterilization of equine endoscopes by wiping with alcohol between patients is less than satisfactory. The current invention is designed as a much more effective and convenient alternative in preventing spread of disease between equine patients, as well as saving the veterinary practitioner the time of attempting to disinfect the endoscope between patients in a less than sufficient manner.

The current methods of obtaining samples for submission to laboratories for culture and sensitivity testing are also less than satisfactory. Often a sample of mucous, for example, is obtained by maneuvering the endoscope in such a manner as to get a sample on the end and hopefully, it will remain there through withdrawal of the endoscope from the patient and can be then collected for submission to a laboratory. Alternatively, endoscope culture swabs may be used which are expensive and involve a complex method of obtaining a sample which is less convenient and takes more time than the present invention. A convenient and cost effective method of obtaining a sample for culture means that more veterinary clients are likely to choose to have a sample collected and submitted to a lab, thus helping veterinarians in their business as well as determining a more accurate diagnosis and therefore treatment plan for their patients.

### SUMMARY OF THE INVENTION

It is an object of the invention to prevent spread of disease between equine patients in an affordable, efficient manner.

It is another object of the invention to easily and efficiently collect a biological sample, such as mucous, for submission to a laboratory for culture.

The present invention is a disposable protective sleeve used for covering equine endoscopes during examination of a patient, in order to help prevent contamination and therefore spread of disease between equine patients. It is comprised of a thin strip of optically clear plastic to form a long protective tube with which to cover the endoscope during use. The plastic is optically clear to allow viewing through the end of the scope while the sleeve is in place. The plastic strip is closed at the distal end, the proximal end being left open and the side edges sealed to form a long tube. The distal end of the endoscope is inserted through the proximal end of the sleeve and continues to be fed through the sleeve until the endoscope is covered by the plastic sleeve. The proximal end of the sleeve may have an elastic piece encircling the circumference of the plastic tube to assist in keeping the sleeve in place on the endoscope. The distal end of the sleeve may or may not also have a small pocket near the end, for collection of samples such as mucous, to collect a sample for laboratory submission and testing, such as for culture and sensitivity for the presence of microorganisms and therefore determining the best method of treatment. Many times, samples are not obtained from equine patients being examined by endoscope, or are obtained in an expensive or inconvenient manner, so providing an easy method for doing such is an additional benefit of the invention and will assist the veterinary practitioner in making a more accurate diagnosis. After an endoscopic examination is completed, the sleeve is pulled off of the endoscope and disposed, and a new protective sleeve is put over the endoscope before a new patient is examined.

This will greatly help to prevent spread of infection between equine patients and will also promote obtaining a culture from an equine patient for proper diagnosis.

### BRIEF DESCRIPTION OF THE DRAWINGS

FIG. 1 shows one embodiment of the invention with the disposable protective sleeve in place over the endoscope, with 1 the proximal end of the sleeve, 2 the distal end of the sleeve, 3 the elastic band at the proximal end of the sleeve, and 4 sample collection pocket near the distal end of the sleeve.

FIG. 2 shows a closer view of the distal end of the disposable protective sleeve with 1 the sample collection pocket near the distal end of the sleeve, and 2 the distal end of the sleeve.

### DETAILED DESCRIPTION OF THE INVENTION

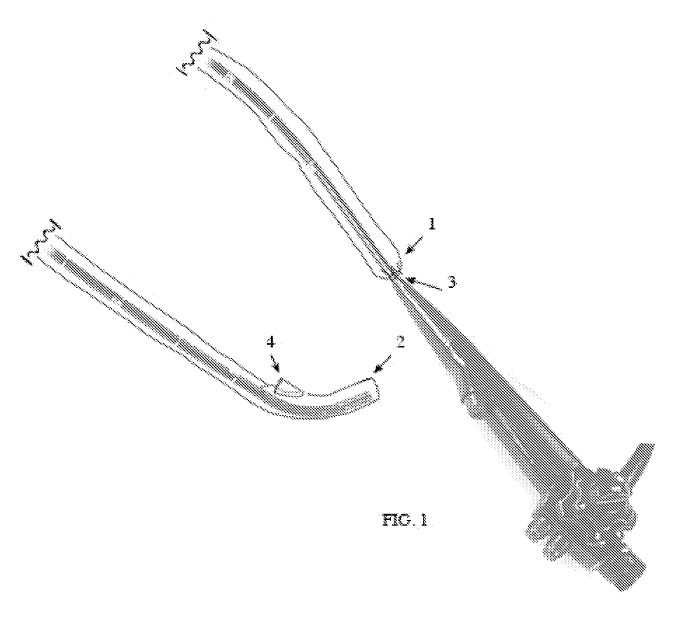

The present invention is a disposable protective sleeve for an equine endoscope. It will be placed over the endoscope by inserting the endoscope through the proximal end of the sleeve (FIG. 1, 1) and feeding the endoscope through until the sleeve is in place. The endoscope can then be used on an equine patient while greatly minimizing contamination to the endoscope and the sleeve disposed after the scope is withdrawn from the patient. The endoscope will then be covered with a new plastic sleeve before use on the next patient.

Oftentimes, endoscope examinations are performed on equines because of illness of the patient, so a better method of preventing spread of disease from one patient to the next is needed. There is usually not time for complete sterilization of the endoscope between equine patients, as this is a lengthy procedure and for best results includes dismantling of the scope to remove all contamination. The process in human medicine involves cleaning of debris, disinfection by immersion in a high-level disinfectant, rinsing with water, and drying. As equine practices are busy and available equipment is more limited than might be the case with human patients, scopes are often cleaned by wiping them with alcohol, a practice which is less than sufficient as well as being inconvenient in adverse conditions, such as cold weather.

The clear plastic sleeve is made to fit equine endoscopes used for common procedures such as but not limited to, examination of the respiratory tract or examination of the stomach. Portable equine endoscopes are usually used for respiratory tract examination and are usually 1 to 1.5 meters in length, while longer endoscopes are used for stomach examination and are generally 3 to 3.5 meters in length. So the endoscope sleeve may be made of different lengths to fit various lengths of equine endoscopes. The clear plastic sleeve may be somewhat loosely fitting around the diameter of the endoscope in order to allow the endoscope to be easily inserted into the sleeve, though the distal end may be somewhat tighter to provide a clear picture from the endoscope.

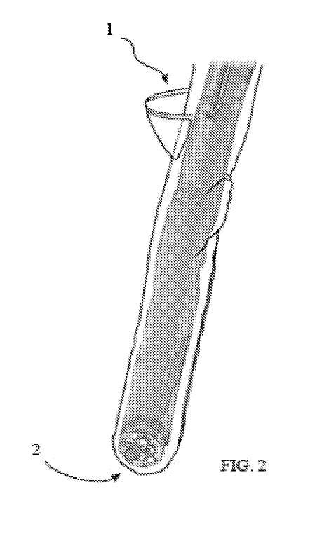

In one embodiment of the invention, the endoscope sleeve will have a small sample collection pocket near the distal end in order to collect a sample, such as mucous, by manipulation of the endoscope by the practitioner (FIG. 1, 4 and FIG. 2, 1). The sample collection pocket will be near enough to the distal end of the endoscope as to be maneuvered into place by the veterinarian via the view from the endoscope. The pocket may be situated on the outside of the tube, or alternately on the inside of the tube with a hole connecting to the outside. Upon removal of the endoscope from the patient, the sample may then be collected from the pocket prior to disposal of the sleeve and the sample then submitted to a laboratory for testing.

The endoscope sleeve may also have an elastic band encircling the diameter of the sleeve at the proximal end, in order to hold the sleeve in place on the endoscope while it is in use during examination of a patient (FIG. 1, 3).

The disposable protective sleeve will be made of clear plastic to enable viewing through the end of the scope with the sleeve in place. It can be manufactured easily from low cost, thin, flexible plastic, using various methods, such as die cutting and heat sealing.

The drawings show one embodiment of the invention, different embodiments of the invention may also be produced without varying from the scope or spirit of the invention.

1. A flexible disposable sleeve made of thin optically clear plastic used for covering an equine endoscope during examination of an equine patient.

2. The endoscope sleeve of claim 1, in which the proximal end is encircled by an elastic band to assist in holding the endoscope sleeve on the endoscope during examination of an equine patient.

3. The endoscope sleeve of claim 1, which has a small collection pocket in the sleeve near the distal end for collection during examination of a biological sample, such as mucous, to be obtained by the practitioner after withdrawal of the endoscope from the equine patient and the sample then submitted to a laboratory for testing, such as for culture and sensitivity.
